(19) United States
(12) Patent Application Publication
(10) Pub. No.: US 2023/0028733 A1
(43) Pub. Date: Jan. 26, 2023

(54) HOOF MUD COMPOSITION

(71) Applicant: Shannon Rosalie Beauclair, Oberlin, LA (US)

(72) Inventor: Shannon Rosalie Beauclair, Oberlin, LA (US)

(21) Appl. No.: 17/443,040

(22) Filed: Jul. 20, 2021

(51) Int. Cl. A61K 38/12; A61K 47/46; A61K 33/34; A61K 33/30; A61K 36/61; A61K 31/4174; A61K 31/7036; A61K 9/06; A61K 9/00

(52) U.S. Cl. CPC A61K 38/12; A61K 47/46; A61K 33/34; A61K 33/30; A61K 36/61; A61K 31/4174; A61K 31/7036; A61K 9/06; A61K 9/0017

(57) ABSTRACT

A therapeutic hoof treatment composition includes clay, copper sulfate, zinc oxide, triple antibiotic ointment, Tea Tree oil, Clotrimazole cream, and water.

### BACKGROUND OF THE INVENTION

The present invention relates to a therapeutic treatment for hoof infections and, more particularly, to a hoof mud composition.

Hoof health is critical to the overall health of a horse. The hoof is vulnerable to microorganisms that cause thrush, central sulcus infection, seedy toe, and white line disease, as well as to puncture wounds and hoof abscesses within the foot. Exposure of healing leg wounds to the elements can cause “proud flesh”. Fungal infections may be introduced, for example, due to scratches on the pastern area of the leg.

Many currently commercially available products do not kill the microorganisms that cause a hoof problem and thus do not help the hoof heal. They also do not form a barrier between the healing hoof and contamination from elements which may get in and reinfect the hoof. Many of these products must be applied daily to work. They do not stay in the hoof long enough to be effective.

As can be seen, there is a need for a composition that treats hoof infections while providing a long-lasting barrier to protect the hoof while it heals.

### SUMMARY OF THE INVENTION

The present invention provides a hoof mud that kills the microorganisms that eat or rot the hoof away and protects the hoof and its infected areas from elements which threaten the integrity of the hoof's overall wellbeing.

In one aspect of the present invention, a therapeutic hoof treatment composition is provided comprising clay, copper sulfate, zinc oxide, triple antibiotic ointment, Tea Tree oil, Clotrimazole cream, and water.

These and other features, aspects and advantages of the present invention will become better understood with reference to the following drawings, description, and claims.

### BRIEF DESCRIPTION OF THE DRAWINGS

FIG. 1 is a perspective view of a horse;

FIG. 2 is a detail top perspective view of a diseased hoof, indicated by line 2-2 of FIG. 1;

FIG. 3 is another detail top perspective view thereof, showing a hoof mud according to an embodiment of the present invention in use on a treatment area;

FIG. 4 is a detail top perspective view of a restored hoof;

FIG. 5 is a bottom perspective view of the diseased hoof;

FIG. 6 is a bottom perspective view showing the hoof mud in use; and

FIG. 7 is a flowchart of a method of treatment according to an embodiment of the present invention.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, one embodiment of the present invention is a therapeutic hoof treatment composition comprising clay, copper sulfate (e.g., powder), zinc oxide, triple antibiotic ointment, Tea Tree oil, Clotrimazole cream, and water.

The inventive hoof mud packs into the crevices of the hoof. Once it is packed in the infected areas, it is then sprayed with water and spread everywhere on the hoof to seal the surface. The inventive hoof mud sticks and stays put, exposing the infected hoof to the active ingredients long enough to fully cure the infection. The mud also protects the hoof from the outside as it heals the hoof from the inside. The hoof then begins to absorb the active ingredients which kill the fungal and bacterial infections. As the hoof absorbs the ingredients, the mud dries and seals the infected pitted areas of the hoof from other debris getting in. As the hoof dampens with the change in weather, the sealed dry clay becomes moist again, releasing more of the active ingredients over days, weeks, and sometimes months.

Clay mixes well with the other ingredients and releases the healing and fungus fighting ingredients slowly as the hoof soaks them in. This clay stays in the pits and holes of the hoof, creating a barrier between it and debris. Clay may be present in the composition in an amount of about 50% by weight to about 60% by weight, such about 53-55% by weight.

Copper sulfate powder is a fungicide commonly used to treat thrush and kills microorganisms which rot the hoof. Copper sulfate may be present in the composition in an amount of about 25% by weight to about 35% by weight, such as about 30-31% by weight.

Water mixes the ingredients together and helps the hoof absorb these fungus fighting agents. The amount of water may be modified to give the mixed ingredients a consistency that adheres to the hoof. Preferably the water is hot, e.g., a temperature in the range of about 90° F to about 140° F. Besides water pre-existing within the individual components, the water content of the composition may be about 5% by weight to about 15% by weight, such as about 9-11% by weight.

Zinc oxide (e.g., 40%) is a mild astringent with weak antiseptic properties that soothes and protects the affected area. It also helps kill hoof decaying microorganisms. Zinc oxide may be present in the composition in an amount of about 1% by weight to about 5% by weight, such as about 3% by weight.

Clotrimazole (e.g., 1%) is an antifungal medication that inhibits growth of microorganisms responsible for hoof rot. Clotrimazole may be present in the composition in an amount of about 0.5% by weight to about 2% by weight, such as about 1% by weight.

Triple antibiotic ointment stops the growth of bacteria and helps kills the infection causing hoof rot while aiding in the healing process. Triple antibiotic ointment may be present in the composition in an amount of about 0.5% by weight to about 2% by weight, such as about 1% by weight.

Tea tree oil is believed to be antibacterial when used topically and helps kill thrush-causing microorganisms. Tea tree oil may be present in the composition in an amount up to about 2% by weight, such as about 1% by weight.

In some embodiments, the weight ratio of copper sulfate to zinc oxide may about 10 to 1.

In some embodiments, the weight ratio of zinc oxide to clotrimazole cream is about 3 to 1.

In some embodiments, the weight ratio of zinc oxide to triple antibiotic ointment is about 3 to 1.

A method of making a hoof mud according to an embodiment of the present invention may include the following steps. About 10 pounds (4536 g) of clay may be broken up into a bowl. The following may be added to the bowl of clay: about 3-4 cups (710 mL-946 mL) of hot water, about 3 cups (2555 g) of copper sulfate, about 8 oz. (227 g) of 40% zinc oxide paste, about 4 ounces (113 g) of triple antibiotic ointment, about 2 ounces (59 mL) of Tea Tree oil, and about 4 ounces (113 g) of Clotrimazole 1% Cream. The ingredients may be blended with a mixer.

A method of treating a hoof with the hoof mud of the present invention may include the following steps. Using a hoof pick, the caretaker may clean out the hoof and make sure it is free of dirt and debris. A spray bottle full of water may help with the cleaning process. This includes pits and separation lines within the hoof wall. An abscess may be treated after it has drained and the hoof is clean. The caretaker may apply one to two tablespoons of mud to the sole of the hoof, working the mud into the infected areas. Once the infected areas have been packed with the clay, the remainder of the mud may be worked into the white line, central sulcus, and collateral grooves of the sole. A layer of mud may be spread evenly over the mud-packed sole. If the mud begins to crumble, the caretaker may apply 1-3 sprays of water to it and continue working it into the hoof. Once completed, the caretaker may hold the hoof up for about an additional minute. The clay dries into the hoof, sealing it from debris as well as from any microorganisms trying to invade the hoof. The ingredients within the mud are slowly released into the hoof.

A hoof mud according to the present invention used twice a week has exhibited a 100 percent success rate for healing central sulcus infections, scratches, white line disease, thrush and seedy toe. Once the hoof is healed, field studies have shown that if applied once a week, the mud prevents the hoof rot conditions from recurring.

In several field studies, seedy toe and white line disease with a depth of approximately one inch healed within six months. The hoof mud has shown great results using it over much less time as well. Central sulcus infection was almost cleared in every case within 6 weeks and minor thrush related separations have also been healed and grown out within six weeks. Scratches along the pastern and lower leg area showed great improvement within weeks and in all cases was completely healed at the 6-week checkups.

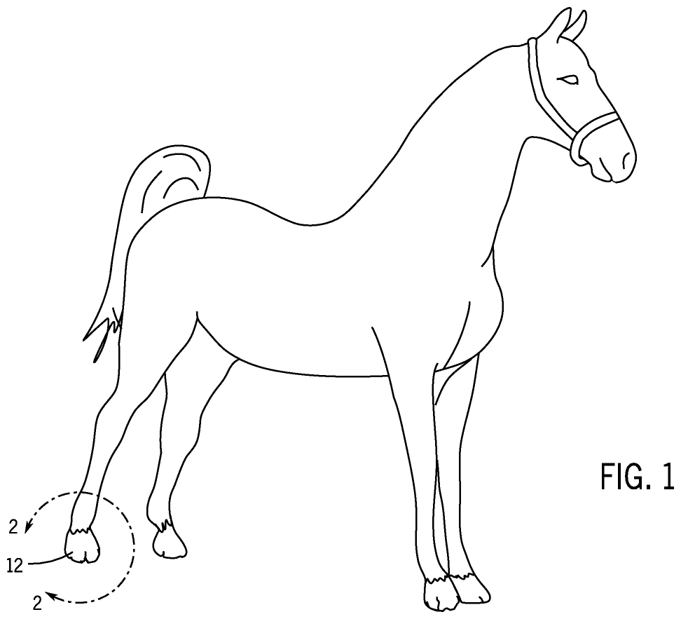

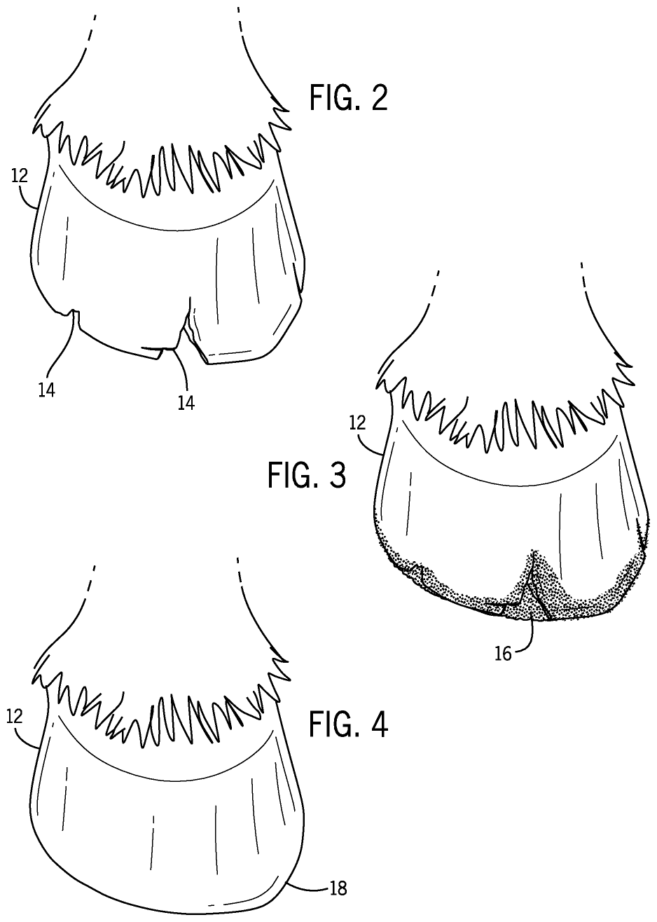

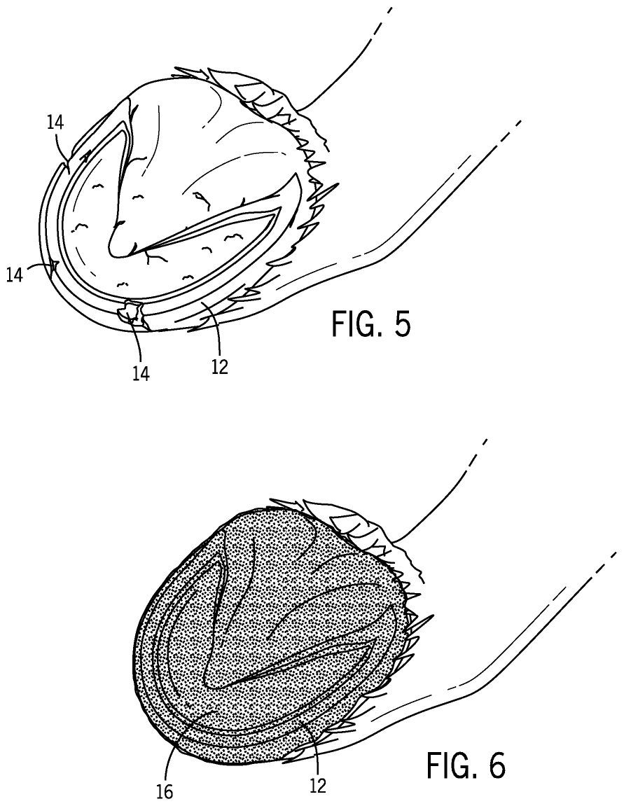

Referring to FIGS. 1 through 7, FIG. 1 shows the location of a hoof 12 to be treated on a horse. FIGS. 2 and 5 illustrate damaged areas 14 of the hoof 12. As shown in FIGS. 3 and 6, the hoof 12 may be packed with the inventive hoof mud 16, treating any infections and allowing the hoof 12 to heal until it is restored 18, as shown in FIG. 4.

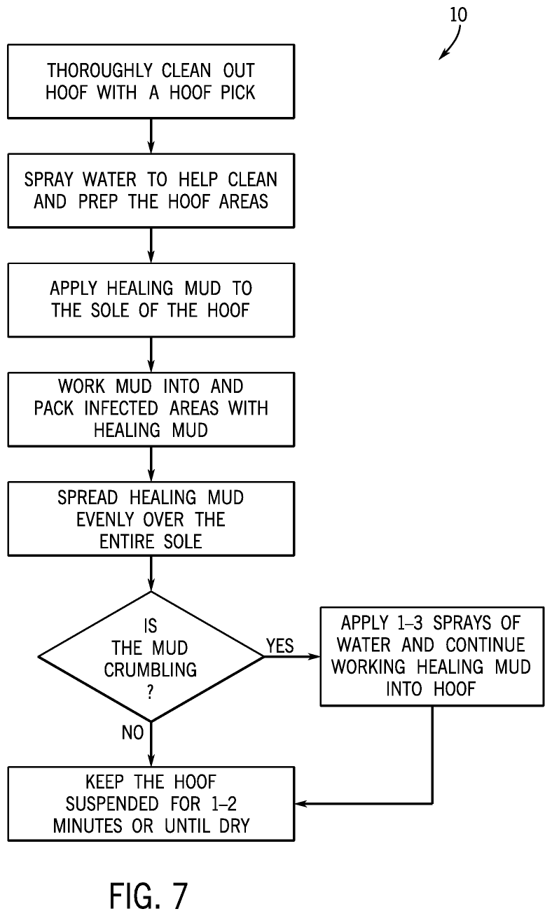

FIG. 7 presents a flowchart 10 illustrating a method of hoof treatment, comprising applying the inventive hoof mud 16. The method includes preparing the hoof for treatment, applying the inventive mud to the hoof, adding water to obtain a suitable consistency, and allowing the mud to set briefly.

It should be understood, of course, that the foregoing relates to exemplary embodiments of the invention and that modifications may be made without departing from the spirit and scope of the invention as set forth in the following claims.

What is claimed is:

1. A therapeutic hoof treatment composition comprising clay, copper sulfate, zinc oxide, triple antibiotic ointment, Tea Tree oil, Clotrimazole cream, and water.

2. The therapeutic hoof treatment composition of claim 1, wherein the clay is present in an amount of about 50% by weight to about 60% by weight.

3. The therapeutic hoof treatment composition of claim 1, wherein the copper sulfate is present in an amount of about 25% by weight to about 35% by weight.

4. The therapeutic hoof treatment composition of claim 1, wherein the zinc oxide is present in an amount of about 1% by weight to about 5% by weight.

5. The therapeutic hoof treatment composition of claim 1, wherein the Clotrimazole cream is present in an amount of about 0.5% by weight to about 2% by weight.

6. The therapeutic hoof treatment composition of claim 1, wherein the triple antibiotic ointment is present in an amount of about 0.5% by weight to about 2% by weight.

7. The therapeutic hoof treatment composition of claim 1, wherein the Tea Tree oil is present in an amount of up to about 2% by weight.

8. The therapeutic hoof treatment composition of claim 1, wherein the water is present in an amount of about 5% by weight to about 15% by weight.

9. The therapeutic hoof treatment composition of claim 1, wherein a weight ratio of copper sulfate to zinc oxide is about 10 to 1.

10. The therapeutic hoof treatment composition of claim 1, wherein a weight ratio of zinc oxide to clotrimazole cream is about 3 to 1.

11. The therapeutic hoof treatment composition of claim 1, wherein a weight ratio of zinc oxide to triple antibiotic ointment is about 3 to 1.
